(19) United States
(12) Patent Application Publication
(10) Pub. No.: US 2020/0054449 A1
(43) Pub. Date: Feb. 20, 2020

(54) TRANSCATHETER MITRAL VALVE

(71) Applicant: Cornell Universrty, Ithaca, NY (US)

(72) Inventors: James K. MIN, Brookyn, NY (US); Bobak MOSADEGH, New York, NY (US); Tracey LUSRIG, New York, NY (US); Simon DUNHAM, New York, NY (US); Eva ROMITO, New York, NY (US); Amir Ali Amiri MOGHADAM, New York, NY (US); Seyedhamidreza ALAIE, New York, NY (US)

(21) Appl. No.: 16/603,523

(22) PCT Filed: Apr. 9, 2018
(86) PCT No.: PCT/US2018/026706

§ 371 Date: Oct. 7, 2019

Related U.S. Application Data
(60) Provisional application No. 62/483,098, filed on Apr. 7, 2017.

(51) Int. Cl. A61F 2/24; A61F 2/848

(52) U.S. Cl. CPC A61F 2/2418; A61F 2/2433; A61F 2/2457; A61F 2002/068; A61F 2220/0016; A61F 2230/0013; A61F 2230/0065; A61F 2/848

(57) ABSTRACT

Transcatheter stent valve implants and implants that preserve functional caliber of the left ventricle outflow tract, LVOT, and streamlined blood flow from left ventricular inflow to the LVOT. The implants have inflatable balloons that prevent regurgitation around the device and allow more accurate sizing and fit.

### RELATED APPLICATIONS

The present application claims priority to, and the benefit of, U.S. Provisional Patent Application No. 62/483,098 entitled “TRANSCATHETER MITRAL VALVE” and filed on Apr. 7, 2017, the entire contents of which are hereby incorporated by reference for all purposes.

### FIELD OF THE INVENTION

The inventions relate to methods, delivery devices and implants for performing transcatheter mitral valve replacements (“TMVR”).

### BACKGROUND OF THE INVENTION

Mitral regurgitation (“MR”) occurs when the native mitral valve is degraded and the leaflets fail to coapt, allowing retrograde flow into the left atrium instead of directing all of the blood in the left ventricle through the aortic valve. An alternative form of MR is in the setting of left ventricular dilatation wherein the dilatation disallows coaptation of the mitral leaflets. In some cases, the coaptation of the native valve leaflets may be reestablished through annuloplasty, which contracts the annulus of the valve to bring the leaflets closer together. Moderate to severe MR may be treated by implanting a prosthetic valve. These prosthetic valves have been historically implanted surgically, and often include resecting a portion of the native valve leaflets. However, at least half of patients requiring mitral valve replacement are not healthy enough to undergo traumatic open-heart surgery, either due to co-morbidities or age.

Over the last few decades, minimally-invasive procedures have been developed for the implantation of prosthetic mitral valves using catheters navigated to the heart through an artery, such as the femoral artery. This form of valve replacement is known as transcatheter mitral valve replacement, commonly, and hereinafter, “TMVR”. TMVR is a highly-specialized technique and is complicated partly by the complex geometry and physiology presented by the mitral valve.

The mitral valve has two leaflets, the anterior leaflet and the posterior leaflet. The posterior leaflet, when closed, covers two-thirds of the valve opening with the anterior leaflet covering the other third. The anterior leaflet makes up for its lack of surface area by rising higher in the valve during systole.

Further complicating the geometry of the mitral valve, the two cusps are surrounded by a fibrous ring known as the mitral valve annulus (“MA”), which has a saddle-like shape known as a hyperbolic paraboloid. The hyperbolic paraboloid includes peaks and valleys that are orthogonal to each other. There are anterior and posterior peaks, and medial and lateral valleys. It has been shown that mitral leaflet stress decreases when the height/width ratio of the peaks and valleys, respectively, are greater than 0.20.

During a cardiac cycle, the geometry of the MA changes, undergoing complex conformational changes that may be described as a combination of two components—sphincteric (circumferential) contraction and annular translation. Optimal TMVR solutions will allow the annulus to maintain both sphincteric contraction and systolic translation in anticipation of LV reverse remodeling. Annular translation takes the form of annular folding, which deepens the “saddle” shape. This translation promotes leaflet coaptation without producing leaflet distortion. This is important because leaflet distortion would limit annular contraction.

Midway through the systolic phase, the MA loses its contraction and dilates. The dilatation is substantially asymmetric due to the fibrous aorto-mitral curtain preventing dilatation in the anterior wall. This results in a flattening of the saddle-shape, which occurs to relieve leaflet stress from modified pressure/volume curve of the left ventricle “LV”, as a result of reduced contractility of the base of the heart.

The cusps of the MV are secured by anchors known as chordae tendinae, or “chords”. There are about 100 chords securing the cusps branching from about 25 major chord trunks. These chords include chordal fibroblasts that are metabolically active. They synthesize a matrix of collagen and elastin, thereby increasing their strength. There are three types of chords—primary, secondary and tertiary.

The primary chords are marginal. They attach to leaflets to maintain leaflet apposition and facilitate valve closure.

The secondary chords are basal. They attach to an undersurface of the leaflets and maintain LV size and geometry. Some of the secondary chords are referred to as “struts” or “stays” and are principal chords attached to the anterior mitral leaflet to maintain papillary annular or ventricular-valvular continuity. They suspend the aorto-mitral angle and maintain a normal LV shape, geometry and function.

The tertiary chords arise from the LV wall and insert only in the posterior leaflet. The exact function of the tertiary chords is unknown.

The MV is located close to the left ventricular outflow tract and the aortic valve, with the geometry of this region of the heart governed by an angle termed the aorto- mitral angle—the angle between the mitral valve and the aorta, is narrowed. In a normal heart, the aorto-mitral angle is about 90 degrees. When the aorto-mitral angle is narrowed, the left ventricular inflow path changes such that inflow occurs along the septum while outflow occurs along the posterior wall. Thus, blood is no longer efficiently directed into the aorta. A TMVR procedure should thus place maintaining a natural aorto-mitral angle as a priority.

The geometry and behavior of the MV is further influenced by the papillary muscles (“PM”), which lie beneath the valve commissures, and generally attach to the middle third of the LV wall. The PMs include helically-arranged fibers which fix the travel distance of the leaflets and prevent prolapse. During diastole, the PMs do not protrude in the LV, and the blood inflow tract forms beneath the open MV leaflets. During systole, the cross-sectional diameter of the PMs increase and encroach into the LV, thereby decreasing the size of the LV inflow tract, and creating an outflow tract between the PMs and the septum known as the Left Ventricle Outflow Tract or “LVOT”.

The MV leaflets are further characterized by rough zones and clear zones. The rough zones are located where the primary chords attach to the free edges of the leaflets. The clear zones are located where the secondary chords attach to the ventricular surface or body of the leaflets.

As stated above, the two leaflets comprising the MV are the anterior or aortic MV leaflet and the posterior or mural leaflet. The anterior leaflet is wider and has a shorter base. It has a smooth surface, free of scallops, and separates the LV inflow and outflow tracts, thereby streamlining the ejection of blood through the LVOT. The height of the anterior leaflets is greater than the height of the posterior MV leaflet.

The posterior (mural) leaflet has a greater circumference, taking up two thirds of the MV. It includes slits or indentations known as scallops. These scallops act like pleats on pants—they expand and contract to accommodate the curved shape of the line of valve closure.

These points are raised because an effective TMVR design should consider the interdependence of the LV free wall and leaflets as the changes in LV geometry may have serious consequences for mitral valve dynamics.

The MV leaflets get bigger with LV dilation, as a compensatory response to prevent MR. This is exacerbated by apical displacement of papillary muscles (due to LV dilation), which alters chordal tethering and reduces MV leaflet coaptation. As such, an effect TMVR design must consider that for patients with functional MR, the anterior mitral leaflet geometry will be altered.

The anterior MV leaflet is dynamic during the cardiac cycle. During diastole, the central portion moves in a direction that has a greater anterior component than a lateral component. During systole, the leaflet maintains a funnel shape—providing a concave surface that faces the LVOT to improve outflow.

The efficacy of the LVOT is often compromised by current TMVR designs for several reasons. For example, the devices often result in a small LVOT diameter; the devices create an LV septal bulge; the devices increase the aorto-mitral angle; the devices protrude into the LV; the devices tend to flare.

### SUMMARY

According to one aspect, the disclosure relates to a cardiac stent valve implant. The cardiac stent valve implant includes a stent body having a first end and a second end, a prosthetic valve closer to the first end of the stent body than the second end of the stent body, a first set of anchoring arms coupled to the first end of the stent body, and a second set of anchoring arms coupled to the second end of the stent body. In some implementations, at least one of the stent body, the first set of anchoring arms, or the second set of anchoring arms is configured such that upon implantation of the device within a native heart valve annulus, at least a portion of the stent body is oriented at an angle with respect to an axis normal to a plane of a native heart valve annulus.

In some implementations, the stent body between the first and second ends has a straight longitudinal axis. In some implementations, the stent body includes a longitudinal axis that is defined by a curved or bent line, resulting in an angled orientation of a portion of the stent body with respect to an axis normal to the plane of the heart valve annulusln some implementations, the first end of the stent body has a cross-sectional shape that is different than the cross-sectional shape of the second end of the stent body. In some implementations, the first end of the stent body has a cross-sectional area that is different than the cross-sectional area of the second end of the stent body.

In some implementations, the prosthetic valve assembly is oriented at an angle with respect to an axis normal to a plane of the native heart valve annulus, thereby preserving the functional caliber of the left ventricular outflow tract.

In some implementations, the anchors of at least one of the first set of anchors or the second set of anchors are arranged asymmetrically about a circumference of at least one end of the cardiac stent valve implant, resulting in an angled orientation of at least a portion of the stent body with respect to the axis normal to the plane of the native heart valve annulus. In some implementations, the asymmetrically arranged anchors have variation in length about the circumference of at least one end of the cardiac stent valve implant, resulting in an angled orientation of at least a portion of the stent body with respect to the axis normal to the plane of the native heart valve annulus. In some implementations, the anchoring arms of at least one of the first or second sets of anchoring arms, before deployment, are substantially straight and only obtain their curved configuration during deployment. In some implementations, the anchoring arms of at least one of the first or second sets of anchoring arms, before deployment, have two curves separating three substantially straight portions of the anchoring arms. In some implementations, the anchoring arms of at least one of the first or second sets of anchoring arms, after deployment, have two curves separating three substantially straight portions of the anchoring arms. In some implementations, the anchoring arms of at least one of the first or second sets of anchoring arms, before deployment, have one curve separating two substantially straight portions of the anchoring arms. In some implementations, the anchoring arms of at least one of the first or second sets of anchoring arms, after deployment, have one curve separating two substantially straight portions of the anchoring arms. In some implementations, the anchors of at least one or more of the second set of anchors, when implanted, anchor the device to an aorto-mitral curtain. In some implementations, the anchors of at least one or more of the second set of anchors, when implanted, anchor the device to a fibrous portion of the native heart valve annulus. In some implementations, the angle of the stent body results from one or more curved portions of one or more of the second set of anchoring arms engaging one or more chordae tendineae.

In some implementations, an exterior surface of the first end of the stent body is coupled to an inflatable toroidal balloon. In some implementations, an exterior surface of the second end of the stent body is coupled to an inflatable toroidal balloon. In some implementations, an exterior surface of the longitudinal axis of the stent body is coupled to an inflatable cylindrical balloon. In some implementations, the inflatable cylindrical balloon includes a honeycombed design. In some implementations, the inflatable cylindrical balloon includes a composite multi-ring design. In some implementations, the inflatable cylindrical balloon includes a composite spiral design.

According to another aspect, the disclosure relates to a cardiac stent valve implant. The cardiac stent valve implant includes a stent body having a first end and a second end, a prosthetic valve closer to the first end of the stent body than the second end of the stent body, a cut-out through the longitudinal portion of the stent body close to the second end of the stent body, a first set of anchoring arms coupled to the first end of the stent body, and a second set of anchoring arms coupled to the second end of the stent body. In some implementations, at least one of the stent body, the first set of anchoring arms, or the second set of anchoring arms is configured such that upon implantation of the device within a native heart valve annulus, at least a portion of the stent body is oriented at an angle with respect to an axis normal to a plane of a native heart valve annulus.

In some implementations, the cut-out is on an anterior face of the second end of the stent body to preserve functional caliber of the left ventricular outflow tract. In some implementations, the cut-out has a u-shape. In some implementations, the cut-out has a polygonal shape. In some implementations, the cut-out has a curved shape. In some implementations, the cut-out is angled between about 30 and 85 degrees with respect to a plane through the second end of the stent body. In some implementations, a point of an intersection of an apex of the cut-out with the anterior aspect of the stent body occurs at a point between the first and second ends of the stent body.

In some implementations, the stent body between the first and second ends has a straight longitudinal axis. In some implementations, the stent body includes a longitudinal axis that is defined by a curved or bent line, resulting in an angled orientation of a portion of the stent body with respect to an axis normal to the plane of the heart valve annulusln some implementations, the first end of the stent body has a cross-sectional shape that is different than the cross-sectional shape of the second end of the stent body. In some implementations, the first end of the stent body has a cross-sectional area that is different than the cross-sectional area of the second end of the stent body. In some implementations, at least a portion of the stent body is configured such that upon implantation of the cardiac stent valve implant within a native heart valve annulus, at least a portion of the stent body is oriented at an angle with respect to an axis normal to the plane of the native heart valve annulus.

In some implementations, the prosthetic valve assembly is oriented at an angle with respect to an axis normal to a plane of the native heart valve annulus, thereby preserving the functional caliber of the left ventricular outflow tract.

In some implementations, the anchors of at least one of the first set of anchors or the second set of anchors are arranged asymmetrically about a circumference of at least one end of the cardiac stent valve implant, resulting in an angled orientation of at least a portion of the stent body with respect to the axis normal to the plane of the native heart valve annulus. In some implementations, the asymmetrically arranged anchors have variation in length about the circumference of at least one end of the cardiac stent valve implant, resulting in an angled orientation of at least a portion of the stent body with respect to the axis normal to the plane of the native heart valve annulus. In some implementations, the anchoring arms of at least one of the first or second sets of anchoring arms, before deployment, are substantially straight and only obtain their curved configuration during deployment. In some implementations, the anchoring arms of at least one of the first or second sets of anchoring arms, before deployment, have two curves separating three substantially straight portions of the anchoring arms. In some implementations, the anchoring arms of at least one of the first or second sets of anchoring arms, after deployment, have two curves separating three substantially straight portions of the anchoring arms. In some implementations, the anchoring arms of at least one of the first or second sets of anchoring arms, before deployment, have one curve separating two substantially straight portions of the anchoring arms. In some implementations, the anchoring arms of at least one of the first or second sets of anchoring arms, after deployment, have one curve separating two substantially straight portions of the anchoring arms. In some implementations, the anchors of at least one or more of the second set of anchors, when implanted, anchor the device to an aorto-mitral curtain. In some implementations, the anchors of at least one or more of the second set of anchors, when implanted, anchor the device to a fibrous portion of the native heart valve annulus. In some implementations, the angle of the stent body results from one or more curved portions of one or more of the second set of anchoring arms engaging one or more chordae tendineae.

In some implementations, an exterior surface of the first end of the stent body is coupled to an inflatable toroidal balloon. In some implementations, an exterior surface of the second end of the stent body is coupled to an inflatable toroidal balloon. In some implementations, an exterior surface of the longitudinal axis of the stent body is coupled to an inflatable cylindrical balloon. In some implementations, the inflatable cylindrical balloon includes a honeycombed design. In some implementations, the inflatable cylindrical balloon includes a composite multi-ring design. In some implementations, the inflatable cylindrical balloon includes a composite spiral design.

According to another aspect, the disclosure relates to a method of implanting a cardiac stent valve implant includes positioning the cardiac stent valve implant within native heart valve annulus, such that at least a portion of the cardiac stent valve implant within a left ventricle of a heart is angled posteriorly with respect to an axis that is normal to a plane of a native heart valve annulus, anchoring the cardiac stent valve implant in place while substantially maintaining the angled orientation of the portion of the cardiac stent valve implant within a left ventricle of a heart, with respect to an axis that is normal to a plane of a native heart valve annulus, and facilitating blood flow through an interior lumen of the cardiac stent valve implant, away from the left ventricular outflow tract.

In some implementations, the method of implanting a cardiac stent valve implant involves anchoring the cardiac stent valve implant with asymmetric anchoring arms that are arranged asymmetrically about a circumference of at least one end of the cardiac stent valve implant. In some implementations, the method of implanting a cardiac stent valve implant involves rotating the cardiac stent valve implant until the asymmetrically arranged anchoring arms are in an appropriate position to yield an angled orientation of a portion of the cardiac stent valve implant.

In some implementations, the method of implanting a cardiac stent valve implant involves orienting the stent valve implant at an angle, with respect to an axis normal to the native heart valve annulus, between about 3 and 45 degrees. In some implementations, the method of implanting a cardiac stent valve implant involves orienting the stent valve implant at an angle, with respect to the axis normal to the native heart valve annulus, between about 3 and 30 degrees. In some implementations, the method of implanting a cardiac stent valve implant involves orienting the cardiac stent valve implant having a stent body with a straight longitudinal axis at an angle, with respect to the axis normal to the native heart valve annulus, and directed away from the left ventricular outflow tract. In some implementations, the method of implanting a cardiac stent valve implant involves positioning a curved or angled cylindrical stent body of the cardiac stent valve implant such that the second end of the stent body is at an angle with respect to an axis normal to the native heart valve annulus, and angles away from the left ventricular outflow tract.

In some implementations, the method of implanting a cardiac stent valve implant involves inflating a balloon at one or more sites on the cardiac valve implant. In some implementations, the method of implanting a cardiac stent valve implant involves positioning the cardiac stent valve implant via a delivery catheter. In some implementations, the method of anchoring a cardiac stent valve implant in place involves retracting and advancing a delivery catheter to promote anchoring into a fibrous aorto-mitral curtain, a fibrous portion of a mitral annulus, or chordae tendineae.

### BRIEF DESCRIPTION OF THE DRAWINGS

These and other aspects, features and advantages of which implementations of the present invention are capable of will be apparent and elucidated from the following description of implementations of the present invention, reference being made to the accompanying drawings, in which:

FIG. 1A is an exploded view of an example implementation of a stent valve implant.

FIG. 1B is an assembled view of an example implementation of the stent valve implant shown in FIG. 1A.

FIG. 2A is a schematic drawing of an example implementation of a stent valve implant having a cylindrical stent body.

FIG. 2B is a sagittal cross-section of a left heart showing an example implementation of the stent valve implant shown in FIG. 2A anchored so that the stent body is tilted posteriorly.

FIG. 2C is a perspective drawing of an example implementation of anchoring arms included within the stent valve implant shown in FIGS. 2A and 2B, having a single curve (elbow) separating two straight portions (upper arm and forearm).

FIG. 2D is a perspective drawing of another example implementation of anchoring arms suitable for inclusion in the stent valve implant shown in FIGS. 2A and 2B, having two curves (elbow and wrist) separating three straight portions (upper arm, forearm, and hand).

FIG. 3A is a schematic drawing of an example implementation of a stent valve implant having a curved stent body.

FIG. 3B is a sagittal cross-section of a left heart showing an example implementation of the stent valve implant shown in FIG. 3A, positioned so that the outflow end of the stent body curves posteriorly.

FIG. 4A is a front elevation of an example implementation of a stent valve implant having a cut-out in the stent body.

FIG. 4B is a sagittal cross-section of a left heart showing an example implementation of the stent valve implant shown in FIG. 4A having a cut-out in the stent body, positioned so that the cut-out faces anteriorly, toward the LVOT.

FIG. 5A is a drawing of vector flow mapping (VFM) from an echocardiogram of a left heart of a patient having a conventional prosthetic mitral valve, the VFM indicating vortex flow in the LV.

FIG. 5B is a drawing of VFM from an echocardiogram of a left heart of a patient having a stent valve implant, the VFM indicating streamlined flow in the LV.

FIG. 6 is a schematic of the aortic valve plane, the plane of the native mitral valve annulus, and the stent valve implant shown in FIG. 2A and 2B, in a patient having the mitral stent valve implant shown in FIGS. 2A and 2B, so positioned that stent body is tilted posteriorly, resulting in a desirable aorto-mitral angle of 90 degrees.

FIG. 7A is a top elevation of an example implementation of a stent valve implant with a first (“inflow”) end having a circular cross-section.

FIG. 7B is a bottom elevation of an example implementation of a stent valve implant with a second (“outflow”) end having an oblong cross-section.

FIG. 7C is a perspective view of an example implementation of the stent valve implant shown in FIGS. 7A and 7B having a curved contour connecting the inflow end having a circular cross-section the outflow end having an oblong cross-section.

FIG. 8A is a perspective view of an example implementation of a stent valve implant within a delivery catheter.

FIG. 8B is a perspective view of an example implementation of a stent valve implant within a delivery catheter and having partially deployed anchoring arms at an outflow end.

FIG. 8C is a perspective view of an example implementation of a stent valve implant partially within a delivery catheter, positioned with an inflow end in the mitral annulus and fully deployed anchoring arms at an outflow end.

FIG. 8D is a perspective view of an example implementation of a stent valve implant having fully deployed anchoring arms at an outflow end and anchoring arms at an inflow end contained within the distal delivery catheter.

FIG. 8E is a perspective view of an example implementation of a stent valve implant having fully deployed anchoring arms at both inflow and outflow ends.

FIG. 8F is a perspective view of an example implementation of a stent valve implant having inflated balloons at both inflow and outflow ends and along the stent body.

FIG. 9A is a perspective view of an example implementation of a stent valve implant having a cylindrical balloon in a honeycomb design.

FIG. 9B is a perspective view of an example implementation of a stent valve implant having a cylindrical balloon in a stacked ring design.

FIG. 9C is a perspective view of an example implementation of a stent valve implant having a cylindrical balloon in a spiral design.

FIG. 10 is a sagittal view of an example implementation of a stent valve implant having a balloon at an inflow end, the balloon having a fill line for inflation via a port, and having a fill plug to maintain inflation attached to a plug tether.

### DETAILED DESCRIPTION

The various concepts introduced above and discussed in greater detail below may be implemented in any of numerous ways, as the described concepts are not limited to any particular manner of implementation. Examples of specific implementations and applications are provided primarily for illustrative purposes.

One of the key challenges in replacing a diseased mitral valve with a valve replacement is the worsening of antegrade LV blood flow patterns, in particular through decreasing the functional caliber of the LVOT. First, conventional prosthetic mitral valves are constructed to direct blood flow along the septum of the LV, creating a vortex in the LV instead of streamlined flow, and thereby decreasing the efficiency with which blood is directed through the LVOT into the aorta. Second, conventional valve replacements decrease the aorto-mitral angle, again causing blood flow to be misdirected through the valve to the septum and creating a vortex that decreases passage of blood through the LVOT. Third, conventional valve replacements may bulge into the LVOT, thereby narrowing the channel, again reducing the efficiency of blood passage into the aorta. Aspects of the following disclosure relate to features of certain stent valve implants that provide for normalized LV blood flow patterns.

An example of how certain stent valve implants improve LV blood flow, and in particular improves LVOT functional caliber, is the implementation whereby the device is directed away from the LVOT toward the posterior LV. In an example implementation of the stent valve implant, the stent body comprises a straight cylinder that may be made of a shape memory alloy, for example nitinol. In another example implementation of the stent valve implant, the stent body comprises a curved cylinder in which the angle formed by the curve may range from 90 to 150 degrees. In another example implementation of the stent valve implant, the stent body comprises a straight or curved cylinder having a cut-out in the anterior aspect of the stent body. In some implementations, the cut-out may have an inverted U-shape. In some implementations, the cut-out may be curved. In some implementations, the cut-out may be polygonal. In some implementations, the cut-out may have a length between about 20 to 50% of the length of the stent body and a width between about 20 to 50% of the diameter of the stent body. In some implementations, the cut-out may have a length of between about 10-20% of the length of the stent body and a width of between about 10 to 20% of the diameter of the stent body. In some implementations, the cut-out may have a length between about 40-70% of the length of the stent body and a width between about 40 to 70% of the diameter of the stent body. In some implementations, a point of intersection of an apex of the cut-out with the anterior aspect of the stent body occurs at a point between the first and second ends of the stent body. In some implementations, the cut-out is angled between about 30 and 85 degrees with respect to a plane through the second end of the stent body. In some implementations, the cut-out is angled between about 30 and 50 degrees with respect to a plane through the second end of the stent body. In some implementations, the cut-out is angled between about 50 and 70 degrees with respect to a plane through the second end of the stent body. In some implementations, the cut-out is angled between about 60 and 85 degrees with respect to a plane through the second end of the stent body.

In some implementations with stent bodies comprising straight cylinders, the stent valve implants are positioned by means of asymmetric anchoring arms at a first “inflow” end or at a second “outflow” end, to result in posterior tilting that directs blood flow away from the LVOT. In some implementations, the tilt angle of the implant with respect to an axis normal to a plane of the native heart valve annulus may range from 3 to 45 degrees. In some implementations, the tilt angle may range from 3 to 20 degrees. In some implementations, the tilt angle may range from 10-30 degrees. In some implementations, the tilt angle may range from 20-45 degrees. In some implementations with stent bodies comprising curved cylinders, the stent valve implants are positioned so that the outflow end angles posteriorly, having angles, with respect to an axis normal to a plane of the native heart valve annulus, ranging from 3 to 45 degrees. In some implementations, the angle of the outflow end may range from 3 to 20 degrees. In some implementations, the angle of the outflow end may range from 10-30 degrees. In some implementations, the angle of the outflow end may range from 20-45 degrees. In some implementations with stent bodies having a cut-out on the anterior aspect of the stent body, the stent valve implant is positioned so that the cut-out faces the LVOT. Implementations of the stent body may be between about 10 to 100 mm in length, and when expanded may be between about 5-50 mm in diameter. In some implementations, the stent body may be between about 10-40 mm in length and when expanded may be between about 5 to 20 mm in diameter In some implementations, the stent body may be between about 50 and 80 mm in length and when expanded may be between about 10 to 30 mm in diameter. Implementations of the stent body may be braided or be sectioned from a longer tube.

In some implementations of the stent valve implant, the tilting of the implant may be accomplished with anchoring arms extending from the outflow end of the stent body. In some implementations of the stent valve implant, there may be 6 or more anchoring arms at the outflow end of the stent body. The arms along the anterior aspect of the outflow of the stent body may be long, between about 50 and 90% of the length of the stent body, or short, between about 25 and 50% of the length of the stent body. In some implementations, the long arms may be between about 60 and 90% of the length of the stent body, and the short arms may be between about 25 and 45% of the length of the stent body. In some implementations, the long arms may be between about 40 and 60% of the length of the stent body, and the short arms may be between about 20 and 30% of the length of the stent body. In some implementations, the longer arms may be primarily on the anterior aspect of the outflow end of the stent body, in order to pull the anterior aspect of the implant inferiorly with respect to the posterior aspect of the implant, whereby the desired posterior tilt is achieved. In some implementations, the anchoring arms at the outflow end of the stent body may have a single curve separating two straight portions (elbow separating forearm and upper arm). In some implementations, the anchoring arms at the outflow end of the stent body may have two curves separating three straight portions (wrist separating hand and forearm, elbow separating forearm and upper arm).

Another approach to improving LV blood flow, and in particular improving LVOT functional caliber, is an example implementation of the stent valve implant that is configured to maintain an aorto-mitral valve angle of 90 degrees. In the example implementations of the stent valve implant disclosed above, the angle at which the stent valve implants are tilted may be chosen so that the normal aorto-mitral angle is preserved.

In some implementations of the stent valve device, the aorto-mitral angle is maintained using inflatable balloons. In some implementations, inflatable balloons may be attached to the inflow end of the implants. In some implementations, inflatable balloons may be attached at both inflow and outflow ends. In some implementations of the stent valve implant, the inflatable balloons may be attached along the length of the stent body, either alone or in combination with balloons at inflow and outflow ends. In some implementations, the balloons at the outflow ends and along the length of the stent body may be fused. In some implementations, the balloon may comprise a polymer such as polyethylene or polyvinyl chloride. In some implementations, the balloon thickness ranges between about 10 and 100 microns when uninflated. In some implementations, the balloon thickness ranges between about 20 and 50 microns. In some implementations, the balloon thickness ranges between about 40 and 60 microns. In some implementations, the balloon thickness ranges between about 50 and 80 microns. In some implementations, the balloon thickness ranges between about 70 and 100 microns. In some implementations, the diameter of the inflated balloon may range from about 1.5 and 6.0 cm. In some implementations, the diameter of the inflated balloon may range from about 1.5 to 2.5 cm. some implementations, the diameter of the inflated balloon may range from about 2.0 to 4.0 cm. In some implementations, the diameter of the inflated balloon may range from about 3.0 to 5.0 cm. In some implementations, the balloons may be inflated with a material that is conformable, such as saline, contrast material, or shear thinning liquid. In some implementations, the balloons may be inflated with less conformable materials, such as hydrogel, epoxy, PMMA, silicone, or polyurethane.

In some implementations of the stent valve implant, the aorto-mitral angle is maintained through specialized manufacture of the prosthetic valve leaflets that are located at the inflow end of the stent valve implant. In some implementations, the prosthetic mitral leaflets are manufactured in a 3D valve area rather than a 2D plane at the level of the trigones to optimize the aorto-mitral angle and maximize the mitral valve cross-sectional area. In some implementations, the prosthetic leaflets may comprise tissue, and sources may be porcine, bovine, equine or homograft. In some implementations, the prosthetic leaflets may be mechanical. In some implementations, there are three leaflets. In some implementations there are two leaflets.

In some implementations, the aorto-mitral angle is maintained through the profile of the stent body. An example implementation of the stent body has a low profile, which improves the aorto-mitral angle.

Another manner in which the described stent valve device improves LV blood flow, and in particular improves LVOT functional caliber, is by preventing device bulge into the LVOT. In an example implementation of the device, there may be the cut-out disclosed above, in the anterior aspect of the device that faces the LVOT, wherein the mass of the implant is minimized at critical contact points with the LVOT. As noted above, in one example implementation, this cut-out may be U-shaped.

Another of the key challenges in replacing a diseased mitral valve with a valve replacement is preventing retrograde flow around the perimeter of the valve replacement, especially as the LV and MA undergo remodeling in the time period following replacement of a diseased valve with a competent valve. A related challenge is preventing damage to the valve apparatus as cardiac contractility improves, producing greater stress on the prosthetic device. Conventional valves generally have a fixed cross sectional area and shape, leading to problems with leakage and impaired MA motion. Further, such valves are not protected from the continual stress of cardiac motion.

The disclosed stent valve implant prevents development of retrograde flow and preserves the sphincteric and translational motions of the MA. In an example implementation of the implant, as previously disclosed, inflatable balloons may surround the inflow end, the outflow end, or the length of the stent body. In some implementations, upon inflation, the balloons allow the implant to continue to provide a tight seal even during increase in MA area of up to 25% and during LV remodeling. In some implementations, the inflatable balloons may comprise polyurethane or silicone that may be embedded with microneedles or micropatterning having outward radiation to enhance the seal. As previously disclosed, inflation of the balloons may be accomplished with many materials, including polymers, silicones, foams, or other substance. Additionally, the balloons protect the device from the stress of continuous cardiac motion in the face of increased LV contractility during remodeling, and may significantly prolong the lifespan of the stent valve implant. In some implementations, using less compliant inflation materials may protect the stent valve implants from high pressure and contractility that is expected from mitral annular contractions. In some implementations, using more compliant inflation materials may allow for “toroidal ballooning” of the stent by dampening the high expected contractile forces. As previously disclosed, a stronger structure may be achieved by using a curable epoxy or polymer, while a more compliant structure may be achieved by use of a hydrogel or liquid.

Specific implementations of the invention will now be described with reference to the accompanying drawings. This invention may, however, be embodied in many different forms and should not be construed as limited to the implementations set forth herein; rather, these implementations are provided so that this disclosure will be thorough and complete, and will fully convey the scope of the invention to those skilled in the art. The terminology used in the detailed description of the implementations illustrated in the accompanying drawings is not intended to be limiting of the invention. In the drawings, like numbers refer to like elements.

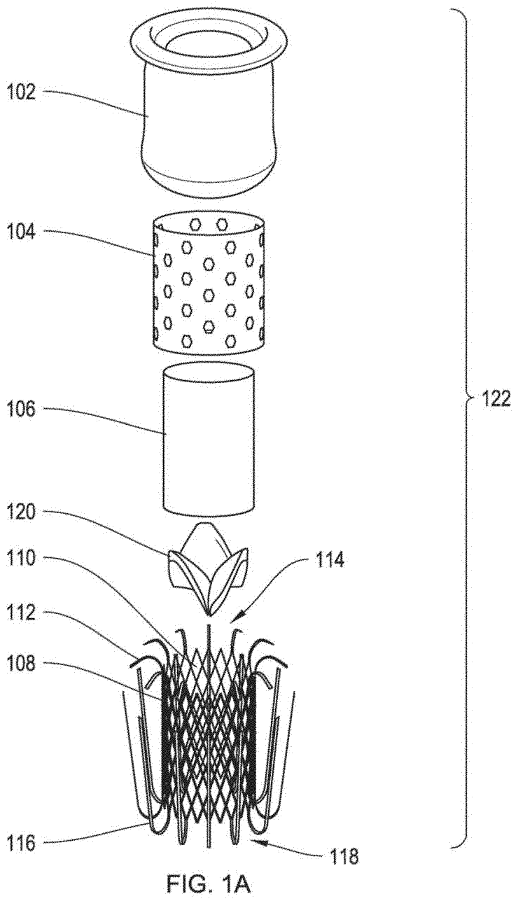

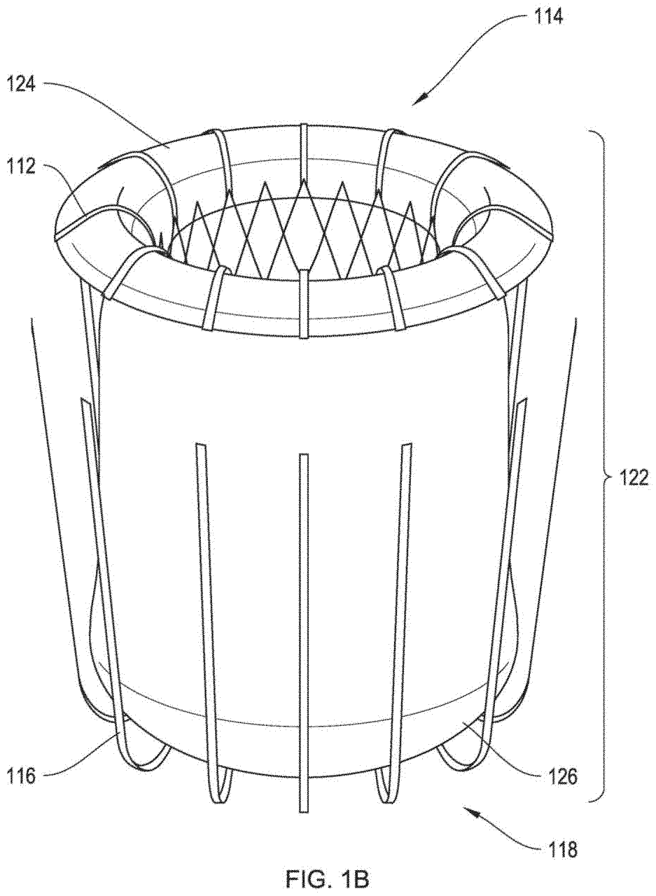

Aspects of the following discussion relate to stent valve implants. These implants may be readily utilized in cardiology and cardiac surgery applications. In particular, left heart physiology may be improved by selecting dimensions, materials, and manufacture for these implants that preserve the LVOT, facilitate streamlined blood flow, and contribute to the longevity of the stent valve implants in vivo. To those ends, components for an example implementation of stent valve implants of this disclosure are discussed in relation to FIGS. 1A and 1B.

FIG. 1A shows an exploded view of a first example implementation of a stent valve implant 122, comprising the following layers, outermost to innermost: a fabric enshroudment 102, a cylindrical balloon 104, an inner fabric layer 106, a stent body 108 from which anchoring arms 112, 116 extend, and a prosthetic valve leaflet assembly 120.

The outermost layer of the first example stent valve implant 122 comprises an enshroudment 102 comprising a material that prevents formation of blood clots and facilitates streamlined blood flow. The material may be synthetic, such as DACRON™. A DACRON™ enshroudment may range from about 0.2 mm to 2.0 mm in thickness. In some implementations, the thickness of the enshroudment may range from about 0.2 mm to 1.0 mm. In some implementations, the thickness of the enshroudment may range from 0.5 mm to 1.5 mm. In some implementations, different synthetic materials may be substituted for DACRON™, such as expanded polytetrafluoroethylene (ePTFE), GORE-TEX™ or polyurethane. In some implementations, the material may be natural, such as porcine small intestinal submucosa (SIS), human allogenic pericardium, or bovine, porcine, or equine xenogenic pericardium.

The layer of the first example implementation of a stent valve, immediately beneath the DACRON™ enshroudment, may be an inflatable “zero thickness” balloon 104. There may be a single balloon cylindrical balloon 104 or multiple balloons. The balloons may be separate or fused. The balloons may comprise a polymer such as polyethylene or polyvinyl chloride. In some implementations, the balloon thickness ranges between 10 and 100 microns when uninflated. . In some implementations, the balloon thickness ranges between about 20 and 50 microns. In some implementations, the balloon thickness ranges between about 40 and 60 microns. In some implementations, the balloon thickness ranges between about 50 and 80 microns. In some implementations, the balloon thickness ranges between about 70 and 100 microns. The advantage of “zero thickness” balloons is the reduction in the diameter of the unexpanded, uninflated implant so that it may be carried in a smaller caliber delivery catheter. In some implementations, the diameter of the inflated balloons may range from about 1.5 to 6 cm. In some implementations, the diameter of the inflated balloon may range from about 4.0 and 6.0 cm. In some implementations, the diameter of the inflated balloon may range from about 1.5 to 2.5 cm. some implementations, the diameter of the inflated balloon may range from about 2.0 to 4.0 cm. In some implementations, the diameter of the inflated balloon may range from about 3.0 to 5.0 cm. In some implementations, the balloons may be inflated with a material that is conformable, such as saline, contrast material, or shear thinning liquid. In some implementations, the balloons may be inflated with less conformable materials, such as hydrogel, epoxy, PMMA, silicone, or polyurethane.

The layer of the first example implementation of a stent valve, immediately beneath the inflatable balloon, is a second fabric layer 106. DACRON™ may be the material selected. Materials including DACRON™ constrain balloon inflation to allow higher filling pressures. In some implementations, different synthetic materials may be substituted for DACRON™, such as ePTFE, GORE-TEX™, or polyurethane. In some implementations, materials may be natural, such as porcine small intestinal submucosa (SIS), human allogenic pericardium, or bovine, porcine, or equine xenogenic pericardium.

The layer of the first example implementation of a stent valve 122, immediately beneath the second fabric layer 106 is a stent body 108 that may be composed of a shape memory alloy such as nitinol. In some implementations, titanium, stainless steel, cobalt-chromium, or platinum chromium may be substituted for nitinol. A stent body 108 may be between about 10 and 100 mm in length, and when expanded between about 5-50 mm in diameter. In some implementations, the stent body 108 may be between about 10-40 mm in length and when expanded may be between about 5 to 20 mm in diameter In some implementations, the stent body 108 may be between about 50 and 80 mm in length and when expanded may be between about 10 to 30 mm in diameter. Implementations of the stent body 108 may be braided or may be sectioned from a longer tube. The stent body 108 may have anchoring arms 112 extending from the inflow end 114 of the implant. The stent body may have anchoring arms 116 extending from the outflow end 118 of the implant.

Within the internal lumen 110 of the stent body 108 of the first example implementation of a stent valve, closer to the first “inflow” end 114 of the stent body than the second “outflow” end 118, is a prosthetic valve leaflet assembly 120. In some implementations, prosthetic leaflets may comprise tissue, and sources may be porcine, bovine, or homograft. In some implementations, the prosthetic leaflets may be mechanical. In some implementations of the prosthetic valve assembly 120, there are three leaflets. In some implementations of the prosthetic valve assembly 120 there are two leaflets.

FIG. 1B shows a perspective view of the components shown in FIG. 1A assembled into the first example implementation stent valve implant (122). In this implementation, anchoring arms 112 extend from the inflow end 114, and anchoring arms 116 extend from the outflow end 118. Additionally shown in FIG. 1B is a toroidal inflatable balloon 124 at the inflow end 114 and a toroidal inflatable balloon 126 at the outflow end 118. As noted above, there may be one, two, or three balloons, and the balloons may be separate or fused.

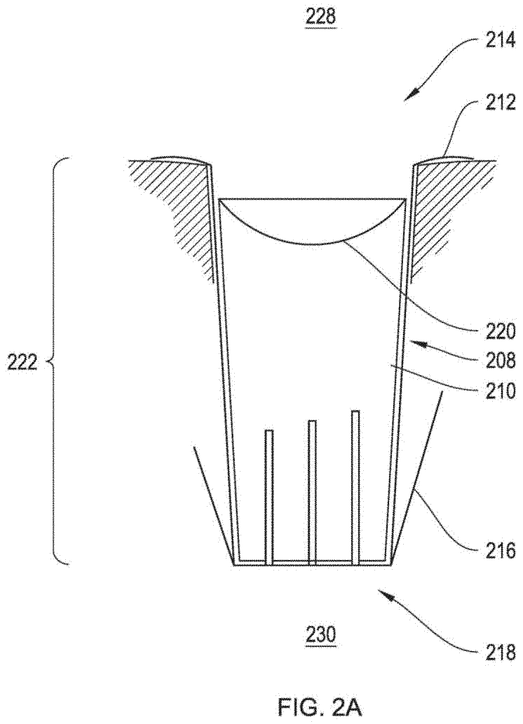

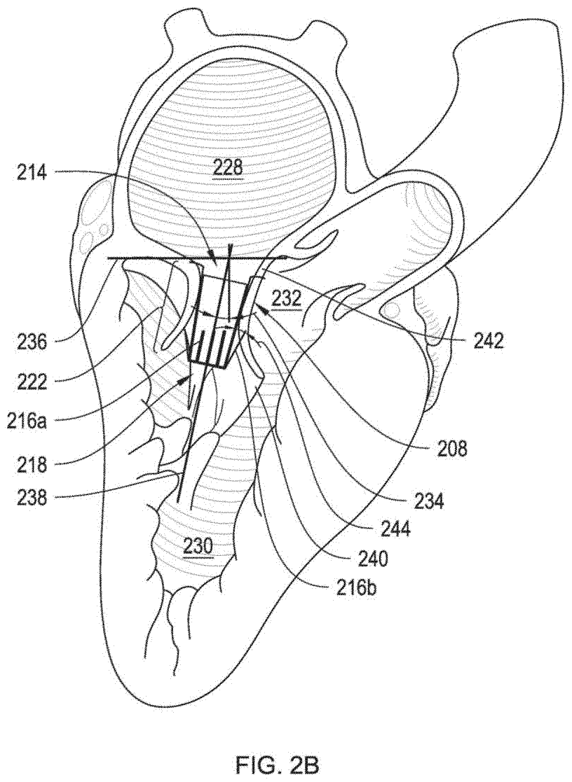

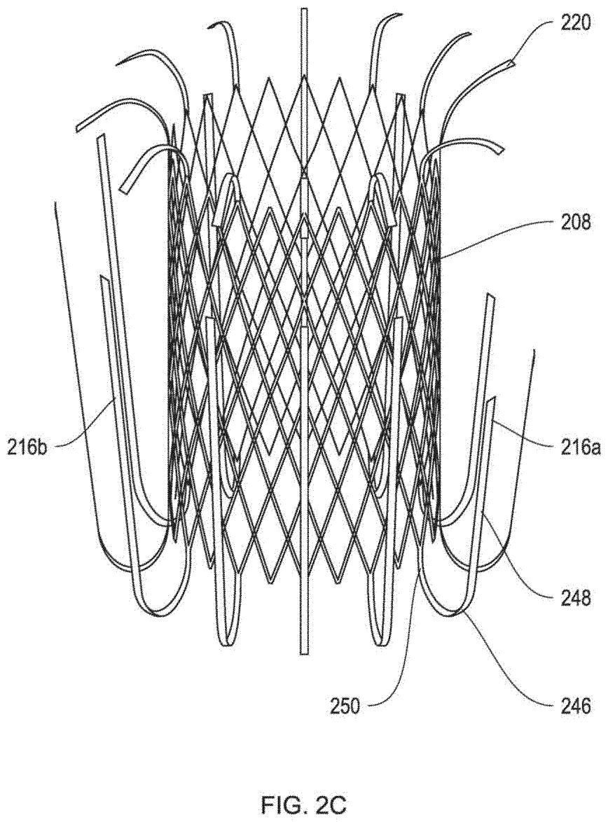

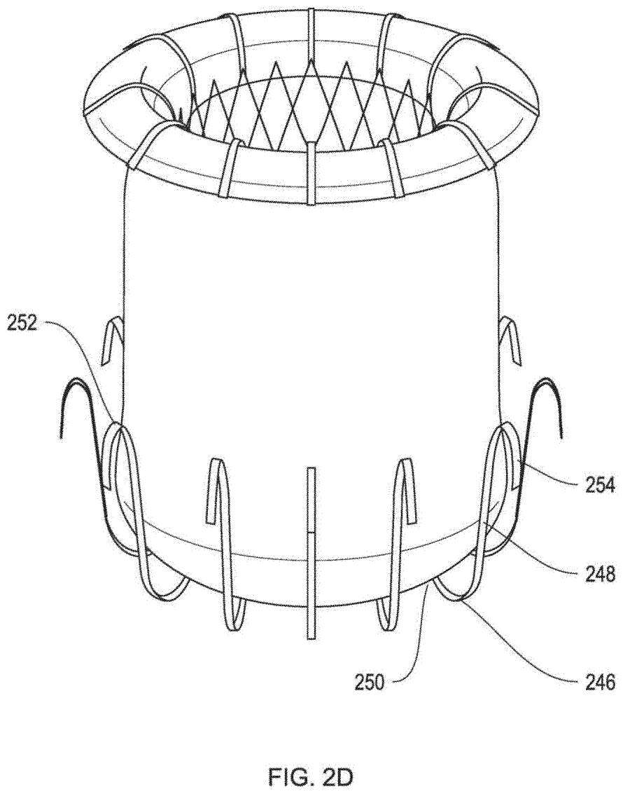

FIGS. 2A-2D show a second example implementation of a stent valve implant 222, comprising a straight cylindrical stent body 210, anchoring arms 212, and valve leaflet apparatus 220.

With reference to the second example implementation of the stent valve implant 222 in FIG. 2A, as implanted in a patient's heart, there may be 6 or more anchoring arms 212 at the inflow end 214. In some implementations, there may be 10 anchoring arms 212 at the inflow end 214. In some implementations, there may be 14 anchoring arms 212 at the inflow end 214. There may be 6 or more anchoring arms 216 at the outflow end 218. In some implementations, there may be 10 anchoring arms 216 at the outflow end 218. In some implementations, there may be 14 anchoring arms 216 at the ouflow end 218. A valve leaflet apparatus 220 may be closer to the inflow end 212 than the outflow end 218 within an internal lumen 210 of the stent body 208. The inflow end 212 may be within the LA 228 of the heart and the outflow end 218 may be in the LV 230 of the heart.

FIG. 2B shows a schematic sagittal view of the left heart of a patient with the second example implementation stent valve implant 222, having a straight cylindrical stent body 208, positioned so that the inflow end 214 may be in the LA 228 of the heart and the outflow end 218 may be in the LV 230 of the heart. The implant may be tilted away from the LVOT (232) of the heart, with the tilt angle 234, with respect to an axis 238 normal to a plane of the native heart valve annulus 236, ranging from about 3 to 45 degrees. In some implementations, the tilt angle 234 may range from 3 to 30 degrees. In some implementations, the tilt angle may range from 5 to 20 degrees. In some implementations, the tilt angle 234 may range from 10-30 degrees. In some implementations, the tilt angle 234 may range from 20-45 degrees. The anchoring arms 216 extending from the outflow end 218 may be asymmetric, with the short arms 216a anchored within the chordae tendineae 240 and the long arms anchored within the aorto-mitral curtain 242. The extended long arms 216b may be between about 50 and 90% of the length of the stent body 208. The extended short arms 216a may be between about 20 and 50% of the length of the stent body 208. In some implementations, the long arms may be between about 60 and 90% of the length of the stent body, and the short arms may be between about 25 and 45% of the length of the stent body. In some implementations, the long arms may be between about 40 and 60% of the length of the stent body, and the short arms may be between about 20 and 30% of the length of the stent body. This implementation accomplishes the tilt angle 234 described above, such that the outflow end 218 is directed posteriorly and away from the LVOT 232, thereby preserving the functional caliber of the LVOT and streamlining blood flow through the LV 230 into the LVOT 232. Anchoring arms 212 at the inflow end 214 may be symmetric or asymmetric.

Asymmetric arms 212 may be selected to help maintain a tilt angle. Long anchoring arms at the inflow end 214 may be 20 to 80% longer than the short anchoring arms on the inflow end 104. In some implementations, anchoring arms of varying lengths 212, 216 may extend circumferentially around one or more ends 214, 218 of the stent body. The length of the stent body 208, as one skilled in the art would understand, is dependent on the patient. However, it is found that a longer stent body 208 for a given patient size may be advantageous over a shorter stent body. This is due, in part, because the anchoring arms 216 associated with a longer stent body 208 may be proportionally longer. The longer the anchoring arm, the smaller the angle 244 produced between the stent body 208 and the anchoring arms 216. A smaller angle 240 may produce stronger anchoring of the stent valve implant 222. Furthermore, a larger angle 240 may produce undesirable greater retrograde force on the stent valve device that may cause the anchor arms 216 to fracture or prolapse.

FIG. 2C shows a schematic of the second example implementation of a stent valve implant 222, having stent body 208, anchoring arms 212 at the inflow end 214 and anchoring arms 216 at the outflow end 218. There may be 6 or more anchoring arms at each end. The anchoring arms 216 extending from the outflow end 218 of the stent body may be asymmetric, as described above. The long arms 216b may predominate on one face of the stent valve implant 222 and the short arms 216a may predominate on the opposite face of the stent valve implant 222. When this second example implementation of a stent valve implant 222 is implanted, the longer anchors may be on the anterior face of the stent valve implant 222, and anchor within the aorto-mitral curtain and fibrous portion of the native heart valve annulus to produce a tilt to the implant such that the outflow end 218 is directed posteriorly and away from the LVOT of the heart. In this implementation, the outflow end anchoring arms 216 have a single curve 246 (referred to as “elbow” 246) separating two straight portions (referred to as “forearm” 248 and “upper arm” 250). In this implementation, the “elbow” facilitates engagement of the stent with internal heart structures, such as the chordae tendineae, providing for the desired tilt.

FIG. 2D shows a schematic of the second example implementation of a stent valve implant 222, but having another embodiment of anchoring arms. In some implementations of the second example implementation of a stent valve device 222, the anchoring arms 216 at the outflow end 218 may be two curved portions (referred to as “elbow” 246 and “wrist” 252) separating three straight portions (“upper arm” 250, “forearm” 248, and “hand” 254). The increase in number of curved and straight portions of the anchoring arms may improve fixation of the implant within a patient's heart relative to the first embodiment of asymmetric anchoring arms described above. In some implementations of anchoring arms that have two curved portions and three straight portions, the anchoring arms may be asymmetric, having long arms and short arms. Fully extended long arms may range in length from 50-90% of the length of stent body 208 and fully extended short arms may range in length from 20-50% of the length of the stent body 208. In some implementations, the extended long arms may be between about 60 and 90% of the length of the stent body, and the extended short arms may be between about 25 and 45% of the length of the stent body. In some implementations, the extended long arms may be between about 40 and 60% of the length of the stent body, and the extended short arms may be between about 20 and 30% of the length of the stent body. Long arms may derive their increased length through enlargement of any one or more of the three straight portions (i.e., “hand,” “forearm,” “upper arm”). In this implementation, the “elbow” and “wrist” facilitate engagement of the stent with internal heart structures, such as the chordae tendineae, providing for the desired tilt.

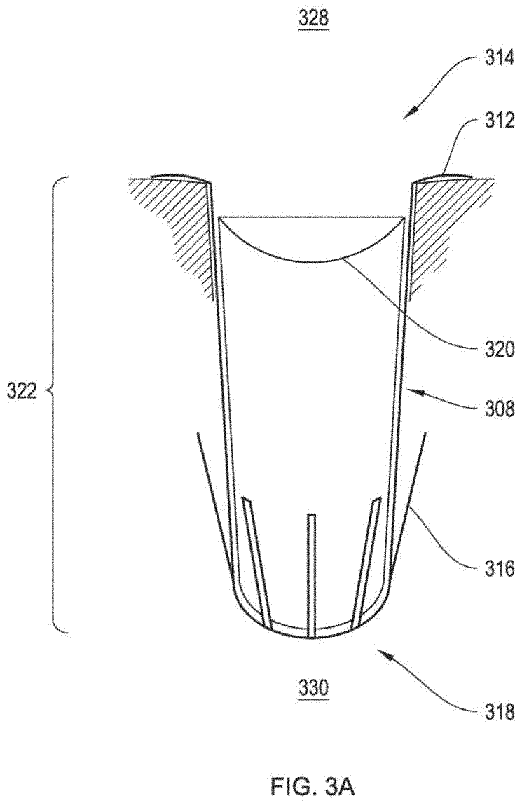

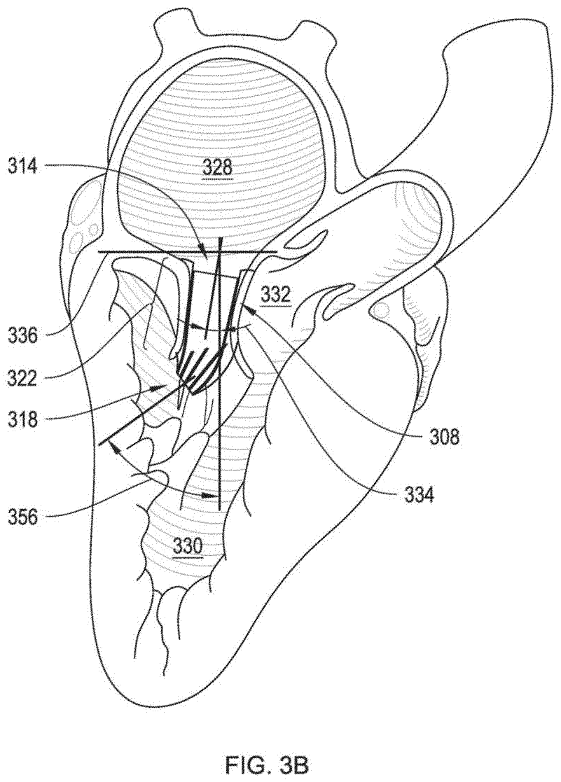

FIG. 3A and 3B show a schematic of a third example implementation of a stent valve implant 322. In the third example implementation, the stent body 308 of may comprise a curved cylinder. The curved cylinder is designed so that the inflow end is angled relative to the outflow end. The third example implementation may share elements with other example implementations, including a prosthetic valve leaflet assembly 320, inflow end anchoring arms 312, and outflow end anchoring arms 316.

With reference to FIG. 3A, showing the third example implementation of a stent valve implant 322 as implanted in a patient's heart, the curve may begin closer to the inflow end 314 than the outflow end 318, at the midpoint between the inflow end 314 and outflow end 318, or closer to the outflow end 318 than the inflow end 314.

FIG. 3B shows a schematic sagittal view of the left heart of a patient in which a third example implementation of a stent valve implant 322, having a curved cylindrical stent body 308, is positioned so that the inflow end 314 may be in the LA 328 of the heart and the outflow end 318 may be in the LV 330 of the heart. The curved stent body 308 has an angle 356 formed by the long axis of the inflow end relative to the long axis of the outflow end. In this implementation, the tilt angle 334 of the implant may range from 3 to 45 degrees. In an implementation when no tilt angle is desired, symmetric anchoring arms 312, 316 extending from outflow 318 or inflow 314 ends of the stent body may be deployed. A tilt angle may be accomplished through deployment of asymmetric anchoring arms 316a, 316b, as described above in FIG. 2B. The angle formed by the curved stent body 356, comprising the right or obtuse angle formed by the intersection of the long axis of the inflow end of the curved stent and the long axis of the outflow end of the curved stent, may range between about 90 to 170 degrees. In some implementations, the angle of the curvature may range between about 90 and 120 degrees, between about 110 and 140 degrees, between about 120 and 150 degrees, or between about 150 and 170 degrees. The outflow end of the third example implementation of a stent valve implant 322, through a combination of tilt angle 334 and angle formed by the curved stent body 308, is directed into the posterior aspect of the LV 330, away from the LVOT 332, thereby preserving the functional caliber of the LVOT 332 and streamlining blood flow through the LV 330 into the LVOT 332.

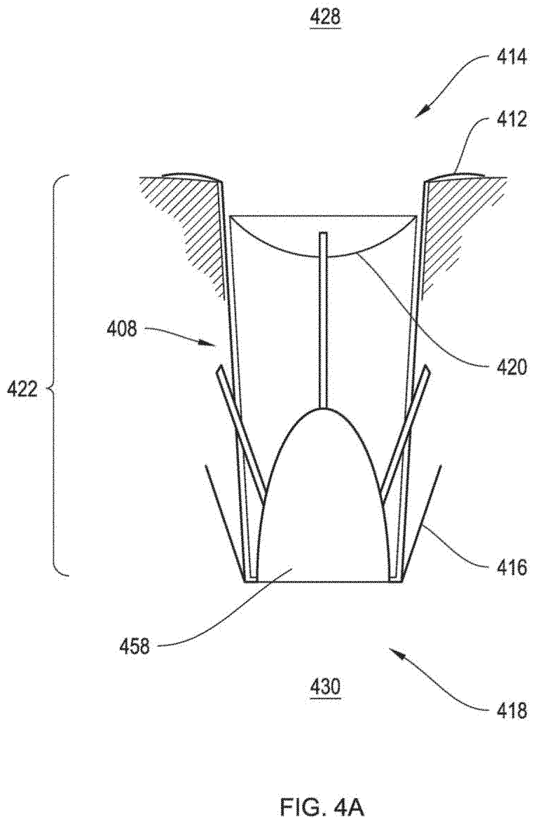

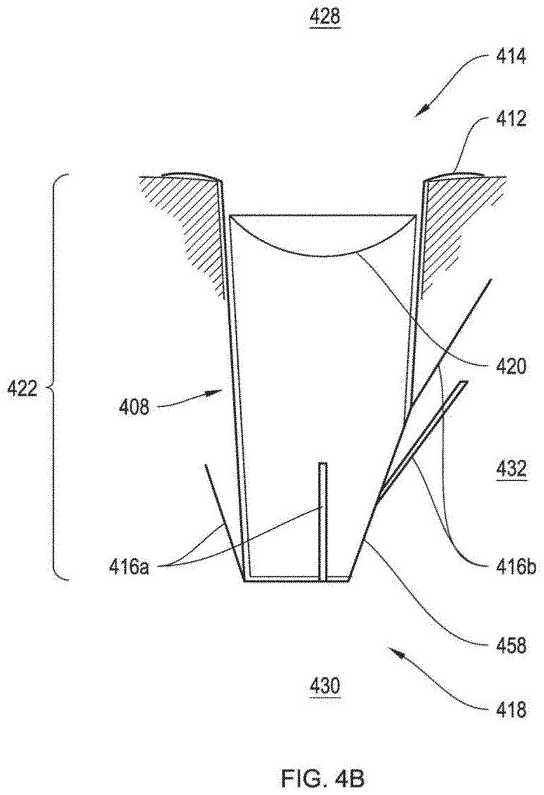

FIG. 4A and 4B show schematics of a fourth example implementation of a stent valve implant 422, having a cut-out 458 on one face of the cylinder, closer to the outflow end 418 than the inflow end 414. The cut-out 458 may have an inverted U-shape that has a length of up to 50% of the length of the stent body and a width of up to 50% of the diameter of the stent body 408. In some implementations, the cut-out may have a length between about 20 to 50% of the length of the stent body and a width between about 20 to 50% of the diameter of the stent body. In some implementations, the cut-out may have a length of between about 10-20% of the length of the stent body and a width of between about 10 to 20% of the diameter of the stent body. In some implementations, the cut-out may have a length between about 40-70% of the length of the stent body and a width between about 40 to 70% of the diameter of the stent body. In some implementations, the cut-out 458 may have the contour of a smooth curve, including a parabola, an oval, or a semicircle. In some implementations, the cut-out 458 may have a polygonal contour, such as a regular polygon or an irregular polygon. In this fourth example implementation of a stent valve implant, the stent body 408 may comprise a straight cylinder or a curved cylinder. In some implementations, the cut-out is angled between about 30 and 85 degrees with respect to a plane through the second end of the stent body. In some implementations, the cut-out is angled between about 30 and 50 degrees with respect to a plane through the second end of the stent body. In some implementations, the cut-out is angled between about 50 and 70 degrees with respect to a plane through the second end of the stent body. In some implementations, the cut-out is angled between about 60 and 85 degrees with respect to a plane through the second end of the stent body.

With reference to FIG. 4A, the fourth example implementation of a stent valve implant 422 may have a stent body 408 that is a straight cylinder. This example implementation may share elements with some implementations, including a prosthetic valve leaflet assembly 420, anchoring arms 412 at the inflow end 414, and anchoring arms 416 at the outflow end 418. Anchoring arms 414, 416 may be all of the same length if no tilt angle 434 is desired, or may be asymmetric, as described in FIGS. 2B and 3B, to achieve a tilt angle 434.

FIG. 4B shows a side elevation of the fourth example implementation of a stent valve implant 422 as positioned in a patient's heart. A cut-out 458 is close to the outflow end 418 and when a stent is implanted, the cut-out may face the LVOT 432. The removal of stent material through the cut-out overcomes a typical problem with valve replacements, whereby a portion of the valve replacements facing the LVOT 432 may bulge into the LVOT 432 and impinge upon the functional caliber of the LVOT 432. In some implementations, the cut-out may have a length between about 20 to 50% of the length of the stent body and a width between about 20 to 50% of the diameter of the stent body. In some implementations, the cut-out may have a length of between about 10-20% of the length of the stent body and a width of between about 10 to 20% of the diameter of the stent body. In some implementations, the cut-out may have a length between about 40-70% of the length of the stent body and a width between about 40 to 70% of the diameter of the stent body. In some implementations, when implanted, it may be positioned to face the LVOT 432 of the heart. The cut-out of this example implementation of a stent valve implant 422 preserves the functional caliber of the LVOT 432 by reducing the surface area of contact between the stent valve implant 422 and the LVOT 432. A prosthetic valve assembly 420 is closer to an inflow end 414. Long anchoring arms 416b are positioned toward the LVOT 432 and short anchoring arms 416a are positioned toward the posterior LV 430 of the heart.

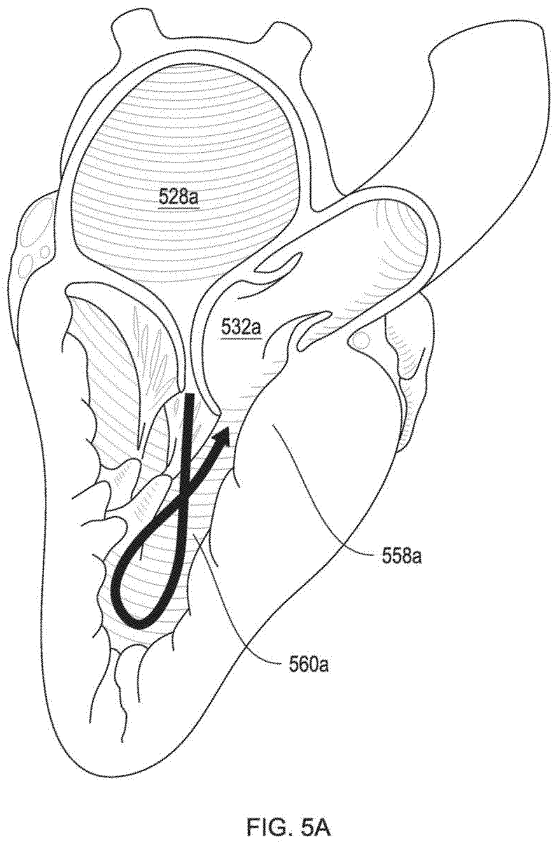

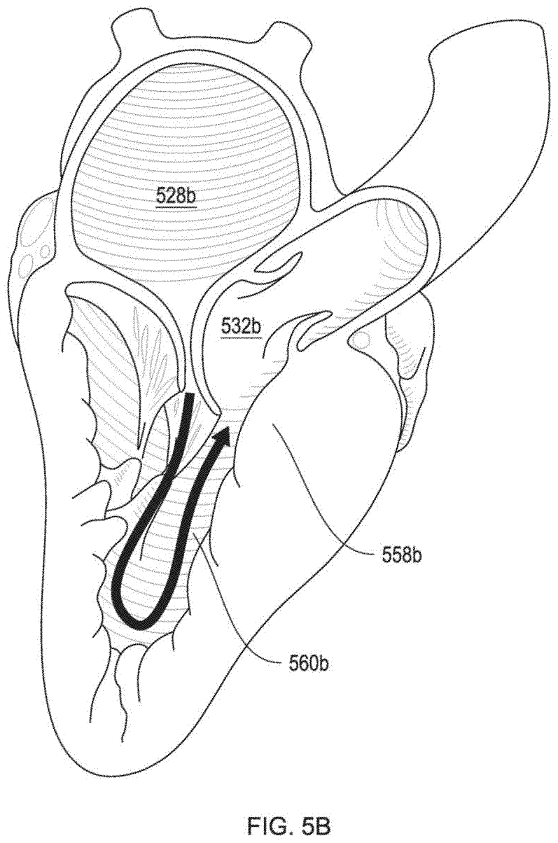

FIG. 5A and 5B are schematics of vector flow mapping (VFM) from echocardiographic studies of two patients with prosthetic mitral valves. VFM is performed during an echocardiographic study to determine whether blood flow within a heart chamber is turbulent or streamlined

FIG. 5A shows a left heart of a patient having a conventional prosthetic mitral valve. VFM demonstrates a blood flow path 560a within the LV 530a of the patient with a conventional prosthetic mitral valve. The blood flow is first directed toward the interventricular septum (IVS) 558, and then creates a vortex 560 indicated by the crossing lines of the flow path 560a. The vortex decreases the efficiency of blood flow through the LVOT 532a, and causes abnormal hemodynamics affecting the function and structure of the LV 530a.

FIG. 5B shows a left heart of a patient having a first example implementation of a stent valve implant 122 in the mitral position. VFM demonstrates a blood flow path 560b within the LV 532b of the heart of the patient with the first example stent valve implant 122. The blood flow is directed toward the posterior wall of the LV 530b, and enters the LVOT 532b in a streamlined fashion, resulting in normal hemodynamics.

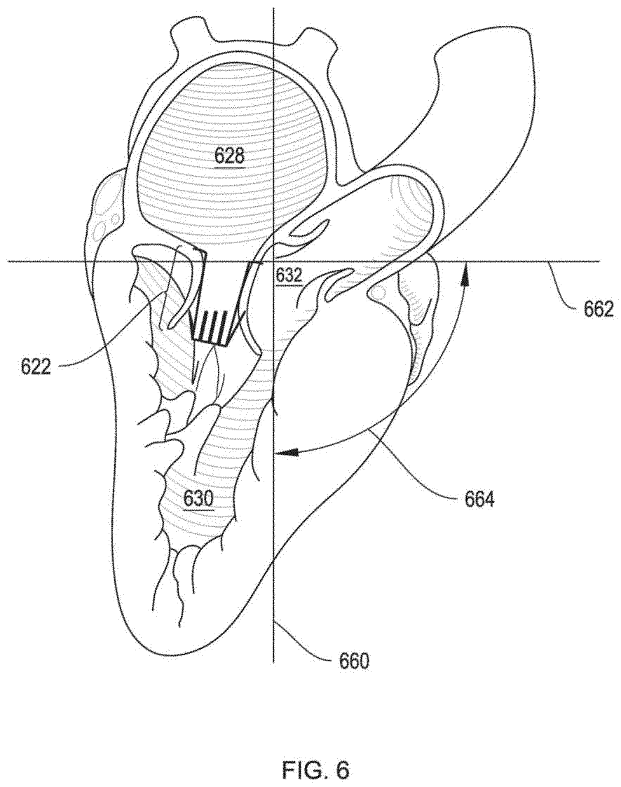

FIG. 6 shows a schematic sagittal view of a left heart of a patient having a second example implementation of a stent valve implant 222 in the mitral position. The aortic valve in this patient forms an aortic plane 660, and the mitral valve in this patient forms a mitral plane 662. The planes' intersection forms a normal aorto-mitral angle 664 of 90 degrees, thereby preserving normal functional caliber of the LVOT 632. In patients having conventional prosthetic mitral valves, the aorto-mitral angle is reduced, reducing the LVOT and resulting in abnormal hemodynamics

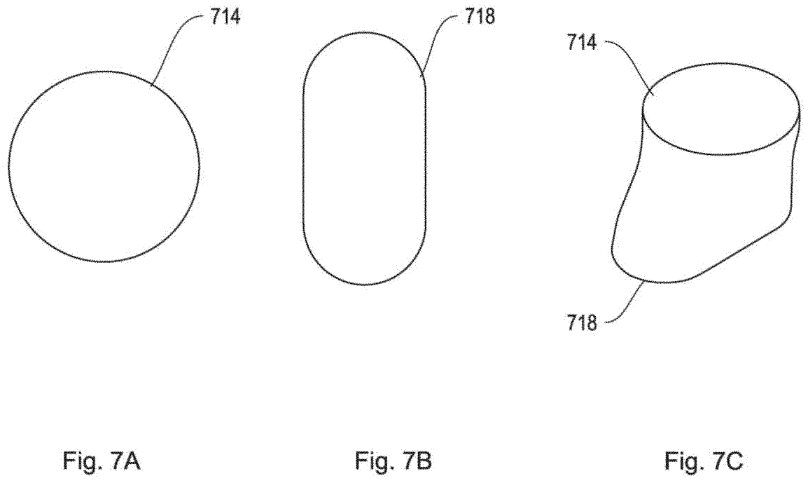

FIGS. 7A-7C show a fifth example implementation of a stent valve implant 722. In this implementation, the implant may have a straight or curved stent, a circular inflow end 714, an oblong outflow end 718, and a curved outer contour 766 connecting the two ends.

With reference to 7A, the fifth example implementation of a stent valve implant 722 has an inflow end 714 that is circular in cross-section. Another example implementation has an inflow end 714 that is D-shaped in cross-section. The goal of circular or D-shaped inflow end 714 is to closely mimic normal mitral valve inflow.

With reference to 7B, the fifth example implementation of a stent valve implant 722 has an outflow end 718 that is oblong in cross-section. Other example implementations have oval or elliptical cross-sections at the outflow end of the implant. The goal of the example implementations described herein is to produce a desirable funnel effect, while maintaining identical cross-sectional areas at inflow and outflow.

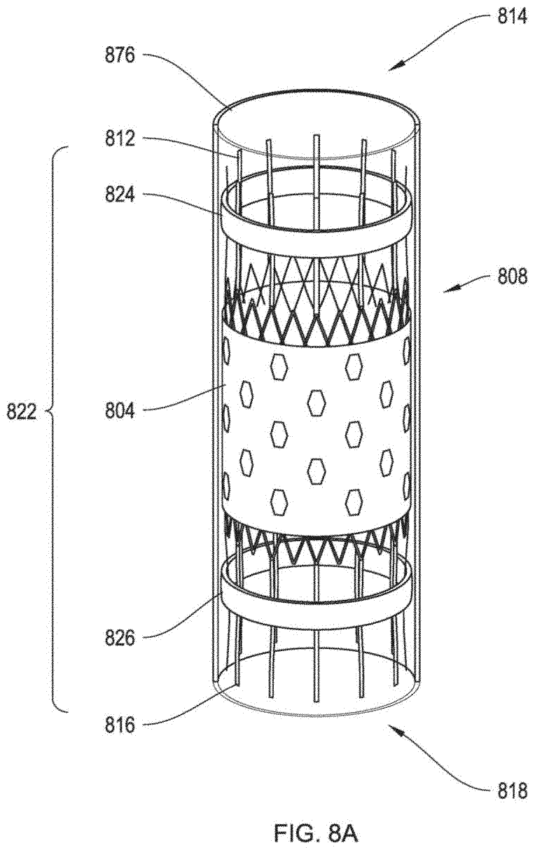

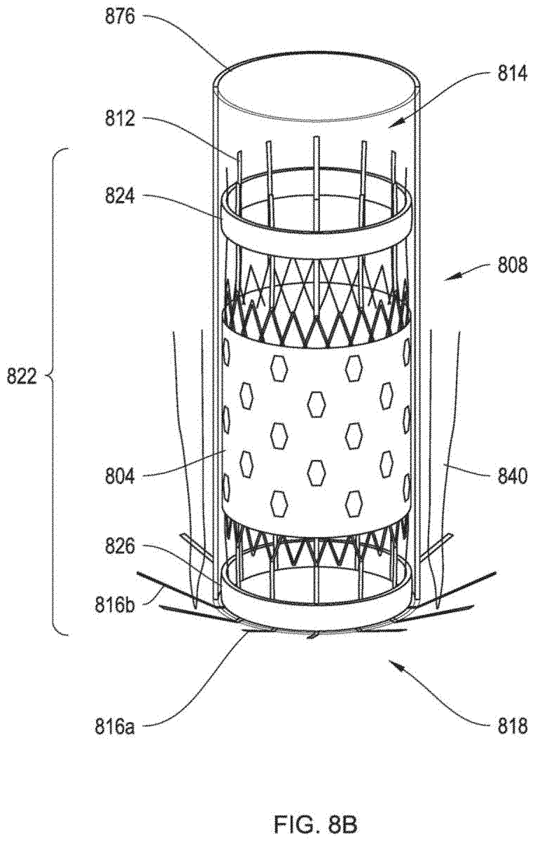

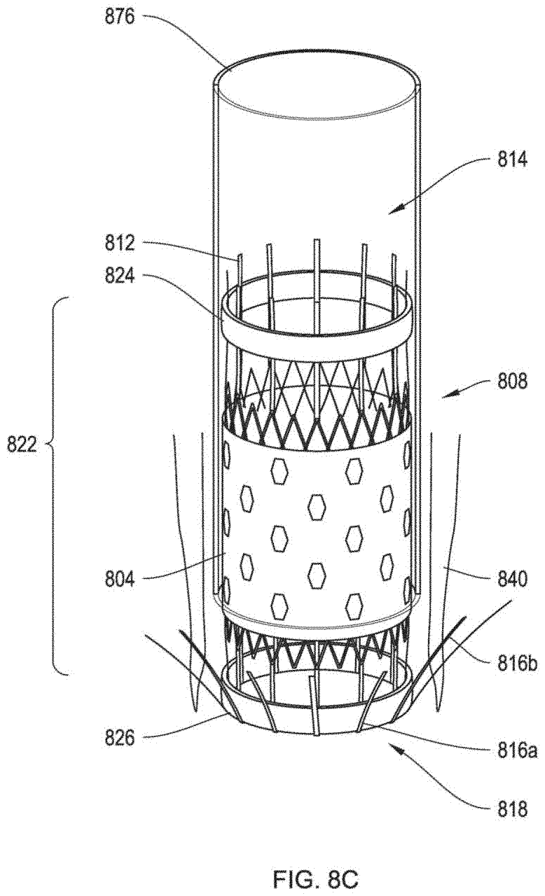

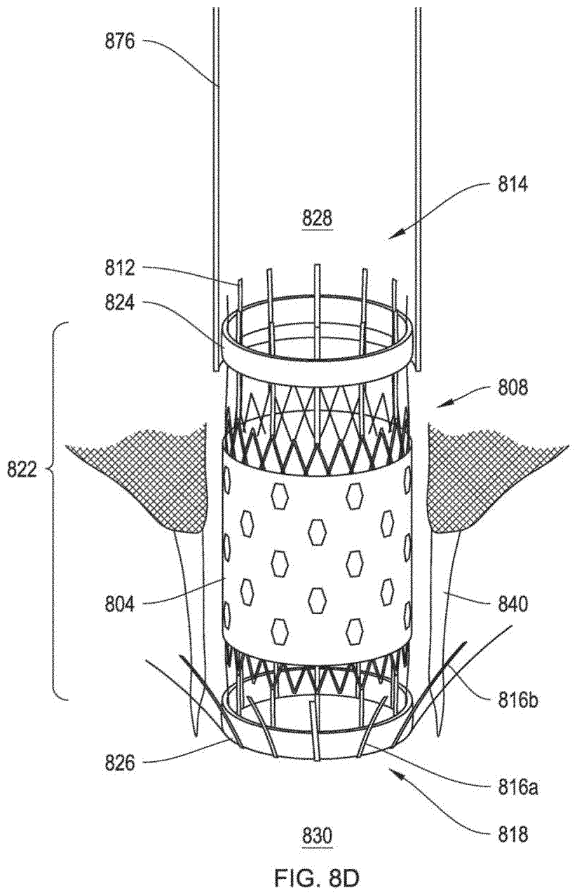

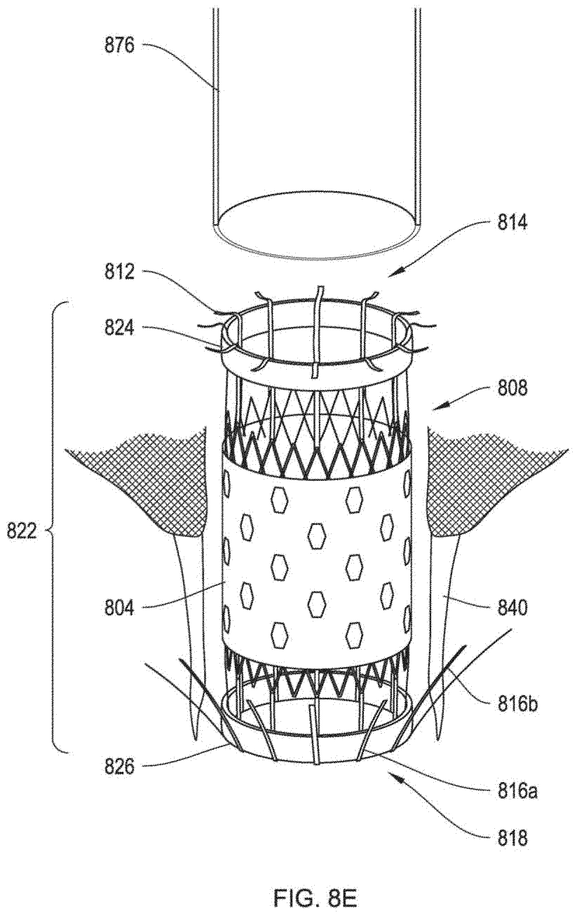

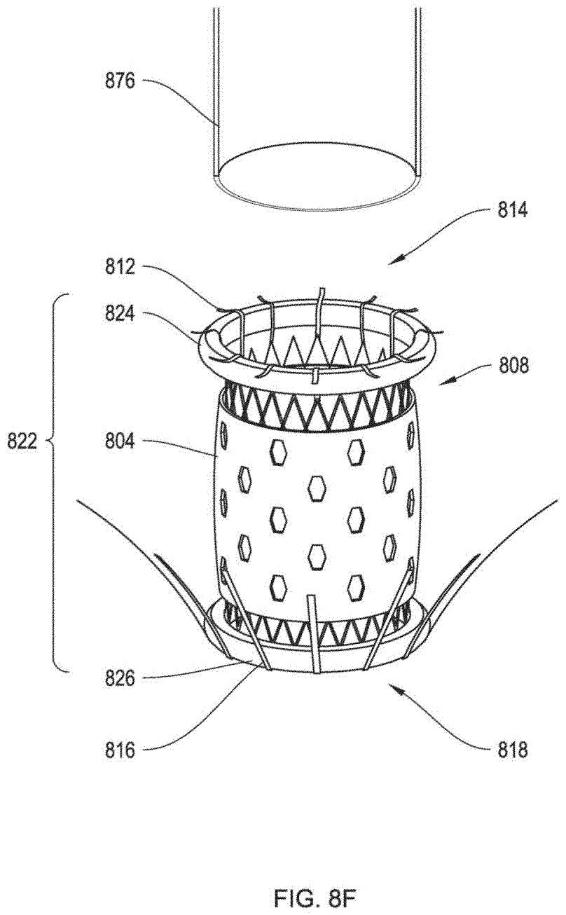

FIGS. 8A-8F are schematics of steps in the implantation of an example implementation of a stent valve implant (822). At the start of implantation, the implant may be contained within a distal end of a delivery catheter that may be between about 8 and 40 F in caliber. In some implementations, a delivery catheter may be between about 10 and 20 F in caliber. In some implementations, a delivery catheter may be between about 20 and 30 F in caliber. A delivery catheter may be introduced via a venous approach, such as the femoral vein, the subclavian vein, or the brachial vein. When a venous approach is used, a delivery catheter may be inserted into the left heart via a transseptal puncture. A delivery catheter may be introduced via an arterial approach, such as the femoral artery, the brachial artery, or the carotid artery. When a venous approach is used, an inflow end of the stent valve implant may be more distally positioned in the catheter than an outflow end. When an arterial approach is used, an outflow end of the stent valve implant may be more distally positioned in the catheter than an inflow end. A delivery catheter may be introduced via a thoracotomy or a sternotomy. When a sternotomy or thoracotomy is used, a delivery catheter may be introduced via a transapical puncture or surgical cardiotomy. Implantation may be guided by imaging, including echocardiography or fluoroscopy, or by direct visualization. Implantation may require guidance of the delivery catheter to a diseased valve in need of repair. Upon positioning near the diseased valve in need of repair, the implantation steps shown in FIGS. 8A-8F may be performed.

FIG. 8A is a schematic of a delivery catheter 876 containing an example implementation of a stent valve implant 822 in its distal end. In this example implementation, the implant may be positioned with an outflow end 818 distal to an inflow end 814. In some implementations, the implant may be positioned with an inflow end 814 distal to an outflow end 818. The implant 822 is unexpanded. There is an uninflated toroidal balloon 824 at the inflow end, an uninflated toroidal balloon 826 at the outflow end, and a cylindrical balloon 804 along the stent body 808. In some implementations, there may be a single balloon, two balloon, or three fused balloons. In this example implementation, a stent body has a straight cylindrical contour 808. Extending from the inflow end 814 of the stent body 808 are anchoring arms 812 that are symmetric. In some implementations, inflow end anchoring arms may be asymmetric, as described in FIGS. 2B and 3B. Extending from the outflow end 818 are anchoring arms 816 that are asymmetric 816a, 816b. In some implementations, outflow anchoring arms may be symmetric, as described in FIGS. 2B and 3B.

FIG. 8B is a schematic of a delivery catheter 876 containing an example implementation of a stent valve implant 822 in its distal end. In this example implementation, anchoring arms 816a,b extending from the outflow end 818 of the stent valve implant 822 are shown partially deployed, indicating retraction and then readvancement of the delivery catheter by an operator, thereby resulting in the outward radial splaying of the outflow end anchoring arms 816a,b. The outflow end anchoring arms in this example implementation are shown to contact chordae tendineae 840. The stent body 808, balloons 804, 824, 826, and inflow end anchoring arms 812 are still contained in the delivery catheter. In some implementations, an operator may rotate the delivery catheter containing the cardiac stent valve implant until the asymmetrically arranged anchoring arms are in an appropriate position to yield a particular angled orientation of a portion of the cardiac stent valve implant.

FIG. 8C is a schematic of a delivery catheter 876 containing an example implementation of a stent valve implant 822 in its distal end. Asymmetric outflow end anchoring arms 816a,b are shown more fully deployed, indicating partial retraction of the delivery catheter 876 by an operator, thereby resulting in fuller extension of the outflow end anchoring arms, and greater contact with surrounding heart tissues. The stent body 808, balloons 804, 824, 826, and inflow end anchoring arms 812 are still contained in the delivery catheter 876.

FIG. 8D is a schematic of a delivery catheter 876 containing an example implementation of a stent valve implant 822 in its distal end. Asymmetric outflow anchoring arms 816a,b are fully deployed and there has been expansion of the stent body 808, indicating more complete retraction of the delivery catheter 876 by an operator, thereby resulting in final positioning of the outflow anchoring arms in surrounding heart tissue, positioning of the outflow end 818 of the expanded stent 808 in the LV 830 of the heart and positioning of the inflow end 814 of the expanded stent 808 in the LA 828 of the heart. Inflow end anchoring arms 812 are still contained in the delivery catheter 876, and balloons 804, 824, 826 are still uninflated. The expanded stent body 808 is positioned so that the inflow end 814 is in the left atrium and the outflow end 818 is in the left ventricle. In some implementations, expansion of a stent body may be accomplished with a coaxial balloon inserted uninflated into the unexpanded stent body and subsequently inflated to produce stent expansion.

FIG. 8E is a schematic of a delivery catheter 876 after full retraction of a delivery catheter 876 and full implantation of a stent valve implant 822. Asymmetric outflow end anchoring arms 816a,b and symmetric inflow end anchoring arms are fully deployed, the former contacting the heart tissues of the left ventricle, and the latter contacting the heart tissues of the left atrium. The expanded stent body 808 is positioned so that the inflow end 814 is in the left atrium and the outflow end 818 is in the left ventricle. Balloons 804, 824, 826 are still uninflated.

FIG. 8F is a schematic of a delivery catheter 876 and an example implementation of a stent valve implant 822, after full retraction of a delivery catheter 876, final positioning of the stent valve implant 822, and inflation of balloons 804, 824, 826. In some implementations, the balloons 804, 824, 826 may be inflated with compliant materials in order to conform readily to surrounding heart tissues, and may continue to conform to surrounding heart tissues as the LV and MA remodel during the time period after implantation of the implant. In some implementations, the balloons 804, 824, 826 may be inflated with less compliant materials in order to create a tight seal and prevent retrograde blood flow. Compliant inflation materials may include saline, contrast material, or shear thinning liquid. Firmer, less compliant inflation materials may include hydrogel, epoxy, polymethyl methacrylate (PMMA), silicone, or polyurethane.

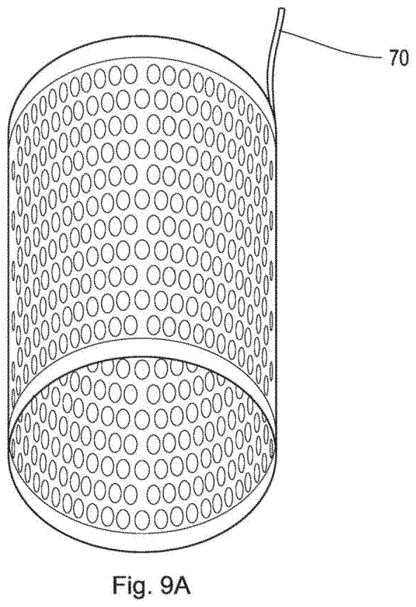

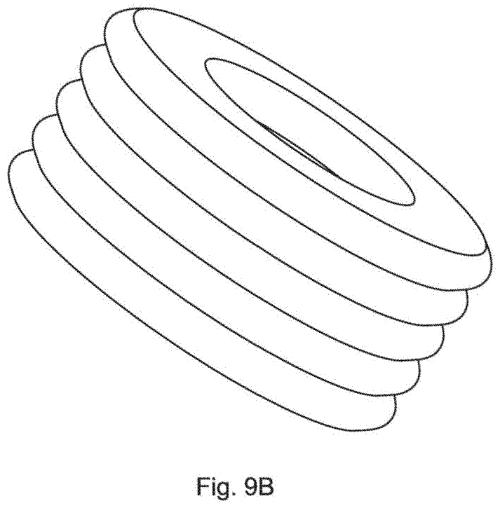

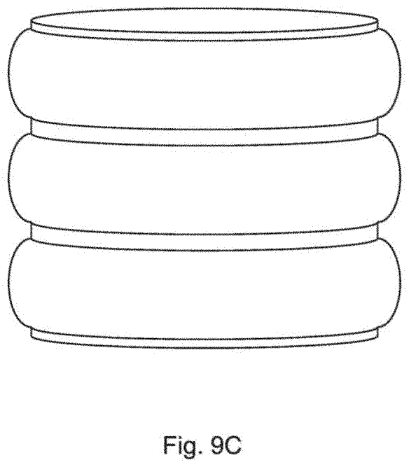

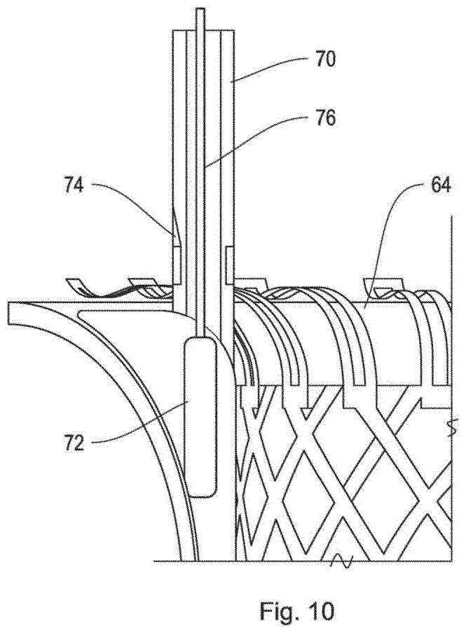

FIGS. 9A-9C are schematics of example implementations of cylindrical balloons with various designs, including honeycomb, multiple ring, or spiral suitable for use with the various stent valve implants described herein. An additional design, shown in FIG. 8, is a plain cylindrical balloon. The plain cylindrical balloon design provides continuous protection of any implementation of a stent valve implant during cardiac contraction. In addition, a plain cylindrical balloon design minimizes stress concentration and has good fatigue performance. Finally, a plain cylindrical balloon design may be “zero volume” at 10-100 microns in thickness when uninflated. In some implementations, the balloon thickness ranges between about 20 and 50 microns. In some implementations, the balloon thickness ranges between about 40 and 60 microns. In some implementations, the balloon thickness ranges between about 50 and 80 microns. In some implementations, the balloon thickness ranges between about 70 and 100 microns. As noted, the “zero volume” characteristic allows an unexpanded, uninflated stent valve device to fit into a smaller caliber delivery catheter.

FIG. 9A is a schematic of any of the example implementations of a cylindrical honeycombed balloon design suitable for use with the various stent valve implants described herein. The advantages of a honeycomb design are resistance to buckling and minimal shrinkage of internal diameter upon inflation. Finally, a balloon with a honeycomb design may be “zero volume” at 10-100 microns in thickness when uninflated. In some implementations, the balloon thickness ranges between about 20 and 50 microns. In some implementations, the balloon thickness ranges between about 40 and 60 microns. In some implementations, the balloon thickness ranges between about 50 and 80 microns. In some implementations, the balloon thickness ranges between about 70 and 100 microns.

FIG. 9B is a schematic of a composite multiple ring balloon design suitable for use with the various stent valve implants described herein. The advantages of a multiple ring design are resistance to buckling, conforming well even to a stent having low density mesh, minimal stress concentration and good fatigue performance. The multiple ring design provides excellent protection against radial forces.

FIG. 9C is a schematic of a composite spiral balloon design suitable for use with the various stent valve implants described herein. The advantages of a composite spiral balloon design are resistance to buckling and having a thickness between 10 and 100 microns when uninflated. In some implementations, the balloon thickness ranges between about 20 and 50 microns. In some implementations, the balloon thickness ranges between about 40 and 60 microns. In some implementations, the balloon thickness ranges between about 50 and 80 microns. In some implementations, the balloon thickness ranges between about 70 and 100 microns.

FIG. 10 is a schematic of an example implementation of a stent valve implant having a balloon at the inflow end 1024 and elements to effect balloon inflation. The inflation elements may include a fill line 1068, through which inflation materials may be instilled. If a more compliant balloon is desired, fill materials may be saline, contrast material, or shear thinning liquid. If a firmer, less compliant balloon is desired, less conformable materials, such as hydrogel, epoxy, PMMA, silicone, or polyurethane, may be used. Other inflation components include the port 1070 through which the fill line is inserted, a fill plug 1072 to assure that the balloon 1024 retains its contents, and a plug tether 1074 to allow precise placement of the fill plug. In some implementations, a balloon may be fitted with a check valve configured to maintain desired pressure within a balloon. By varying the amount and choice of inflation materials, a balloon may be sized to assure that no retrograde flow occurs around the perimeter of the stent valve implant. Furthermore, the balloon allows for remodeling of the MA and LV following placement of a stent valve implant. In some implementations, the diameter of the inflated balloon may range from about 4.0 and 6.0 cm. In some implementations, the diameter of the inflated balloon may range from about 1.5 to 2.5 cm. some implementations, the diameter of the inflated balloon may range from about 2.0 to 4.0 cm. In some implementations, the diameter of the inflated balloon may range from about 3.0 to 5.0 cm.

Although the invention has been described in terms of particular implementations and applications, one of ordinary skill in the art, in light of this teaching, may generate additional implementations and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1. A cardiac stent valve implant comprising:
a stent body having a first end and a second end;
a prosthetic valve assembly coupled to an interior surface of the stent body closer to the first end than to the second end;
a first set of anchoring arms coupled to the first end of the stent body; and
a second set of anchoring arms coupled to the second end of the stent body, wherein at least one of the stent body, the first set of anchoring arms, or the second set of anchoring arms are configured such that upon implantation of the device within a native heart valve annulus, at least a portion of the stent body is oriented at an angle with respect to an axis normal to a plane of a native heart valve annulus.

2. The device of claim 1, wherein the anchors of at least one of the first set of anchors or the second set of anchors are arranged asymmetrically about a circumference of at least one end of the cardiac stent valve implant, resulting in an angled orientation of at least a portion of the stent body with respect to the axis normal to the plane of the native heart valve annulus.

3. The device of claim 2, wherein the asymmetrically arranged anchors have variation in length about the circumference of at least one end of the cardiac stent valve implant, resulting in an angled orientation of at least a portion of the stent body with respect to the axis normal to the plane of the native heart valve annulus.

4. The device of claim 1, wherein the stent body between the first and second ends has a straight longitudinal axis.

5. The device of claim 1, wherein the stent body has a longitudinal axis that is defined by a curved or bent line, resulting in an angled orientation of a portion of the stent body with respect to an axis normal to the plane of the native heart valve annulus.

6. The device of claim 1, wherein the first end of the stent body has a cross section that is different than a corresponding cross section of the second end of the stent body.

7. (canceled)

8. The device of claim 1, wherein the anchoring arms of at least one of the first or second sets of anchoring arms, before or after deployment, have two curves separating three substantially straight portions of the anchoring arm.

9. (canceled)

10. The device of claim 1, wherein the anchoring arms of at least one of the first or second sets of anchoring arms, before or after deployment, have one curve separating two substantially straight portions of the anchoring arms.

11. (canceled)

12. The device of claim 1, wherein an exterior surface of the first end or the second end of the stent body is coupled to an inflatable toroidal balloon.

13. (canceled)

14. The device of claim 1, wherein an exterior surface of the longitudinal axis of the stent body is coupled to an inflatable cylindrical balloon, and wherein the inflatable cylindrical balloon comprises a honeycombed design, a composite multi-ring design, or a composite spiral design.

15. (canceled)

16. (canceled)

17. (canceled)

18. The device of claim 1, wherein the anchors of at least one or more of the second set of anchors, when implanted, anchor the device to an aorto-mitral curtain or to a fibrous portion of the native heart valve annulus.

19. (canceled)

20. The device of claim 1, wherein the prosthetic valve assembly is oriented at an angle with respect to an axis normal to a plane of the native heart valve annulus, thereby preserving the functional caliber of the left ventricular outflow tract.

21. The device of claim 1, wherein the angle of the stent body results from one or more curved portions of one or more of the second set of anchoring arms engaging one or more chordae tendineae.

22-48. (canceled)

49. A method of implanting a cardiac stent valve implant, comprising:
positioning the cardiac stent valve implant within native heart valve annulus, such that at least a portion of the cardiac stent valve implant within a left ventricle of a heart is angled posteriorly, with respect to an axis that is normal to a plane of a native heart valve annulus; and
anchoring the cardiac stent valve in place while substantially maintaining the angled orientation of the portion of the cardiac stent valve implant within a left ventricle of a heart, with respect to an axis that is normal to a plane of a native heart valve annulus; and
facilitating blood flow through an interior lumen of the cardiac stent valve implant, away from a left ventricular outflow tract.

50. The method of claim 49, wherein anchoring the cardiac stent valve implant comprises anchoring the cardiac stent valve implant with asymmetric anchoring arms that are arranged asymmetrically about a circumference of at least one end of the cardiac stent valve implant.

51. The method of claim 50, wherein positioning the cardiac stent valve implant in the native heart valve annulus comprises rotating the cardiac stent valve implant until the asymmetrically arranged anchoring arms are in an appropriate position to yield an angled orientation of the portion of the cardiac stent valve implant.

52. The method of claim 49, wherein the portion of the cardiac stent valve implant oriented at an angle with respect to the axis normal to the native heart valve annulus is positioned at an angle of between about 3 and 45 degrees relative to the axis.

53. The method of claim 49, wherein the portion of the cardiac stent valve implant oriented at an angle with respect to the axis normal to the native heart valve annulus is positioned at an angle of between about 3 and 30 degrees relative to the axis

54. The method of claim 49, wherein positioning the cardiac stent valve implant within the native heart valve annulus comprises positioning a straight cylindrical stent body of the cardiac stent valve implant within the native heart valve annulus at an angle with respect to an axis normal to the native heart valve annulus, and directed away from the left ventricular outflow tract.

55. The method of claim 49, wherein positioning the cardiac stent valve implant within the native heart valve annulus comprises positioning a curved or angled cylindrical stent body of the cardiac stent valve implant such that the second end of the stent body is at an angle with respect to an axis normal to the native heart valve annulus, and angles away from the left ventricular outflow tract.

56. The method of claim 49, wherein the implantation includes inflating a balloon at one or more sites on the cardiac valve implant.

57. The method of claim 49, wherein positioning the cardiac stent valve implant comprises positioning the cardiac stent valve implant via a delivery catheter.

58. The method of claim 49, wherein anchoring the cardiac stent valve in place comprises retracting and advancing a delivery catheter to promote anchoring into a fibrous aorto-mitral curtain, a fibrous portion of a mitral annulus, or chordae tendineae.
